(19) United States
(12) Patent Application Publication
(10) Pub. No.: US 2018/0014969 A1
(43) Pub. Date: Jan. 18, 2018

(54) ASSISTIVE DEVICE FOR REMEDIATING INFLAMMATION, SWELLING AND PAIN

(71) Applicant: Raymond K. WURAPA, Blacklick, OH (US)

(72) Inventor: Raymond K. WURAPA, Blacklick, OH (US)

(21) Appl. No.: 15/651,636

(22) Filed: Jul. 17, 2017

Related U.S. Application Data
(60) Provisional application No. 62/362,933, filed on Jul. 15, 2016.

(51) Int. Cl. A61F 7/02; A61F 7/00

(52) U.S. Cl. CPC A61F 7/02; A61F 2007/0292; A61F 2007/0242; A61F 2007/0238; A61F 2007/0036; A61F 2007/0209; A61F 2007/0204; A61F 2007/0219

(57) ABSTRACT

Anatomically contoured assistive devices, particularly for extremities such as the hand, are form fitting to the tissue to ensure targeted delivery of therapeutic treatment to specific tissue, remain stably affixed without pinching or binding, and allow for motion and use of the tissue while delivering targeted therapeutic treatment.

### RELATED APPLICATIONS

This application claims the benefit under 35 U.S.C. §119(e) to U.S. Provisional Application Ser. No. 62/362,933 filed Jul. 15, 2016, the entirety of which is incorporated herein by reference.

### FIELD OF INVENTION

The invention relates generally to an assistive device for remediating one or more of inflammation, swelling and pain, in particular in the hand and wrist. The assistive device is particularly suitable for continuous wear, and adaptable for providing a plurality of modalities of therapy.

### BACKGROUND

Pain associated with inflamed joints affects a wide swath of the population. Arthritic pain is particularly problematic because it is chronic and persistent, and requires significant ongoing care. Arthritis is a common condition affecting more than 50 million adults in the United States. The hands and wrists are frequently involved, with symptoms including swelling, pain, and joint stiffness. In addition, there are approximately 3.5 million hand and wrist injuries annually in the United States seen at emergency facilities. Many of these injuries are minor, and mainly require cyclic cold application, as well as immobilization and elevation to minimize swelling/tissue inflammation and expedite healing.

Generally, in the context of joint, bone and associated soft tissue discomfort, a wide variety of non-surgical treatments abound beyond drugs and topical medications. Therapy involving application of heat or cooling, as described above, can play a significant role in helping manage these symptoms for acute care post-injury and post-surgery, and for chronic conditions, such as arthritis. Assistive devices in the form of pads and packs deliver warmth or cooling to provide temporary relief to painful and swollen joints. But these treatments can be bulky and cumbersome, are often prone to leakages, and typically cannot be readily held in place to allow the user freedom to engage in typical day-to-day, professional, and recreational activities. This is particularly the case with treatments for extremities such as the hands and wrists.

There are examples in the art of devices for delivery of cooling or warmth that are adapted to be affixed to or donned by a user, such as mitts and mittens. But these assistive devices typically suffer from many of the same challenges of pads and packs in that they are bulky and cumbersome, and though they may be held in place by virtue of fitting over the tissue, such as the hand, they generally interfere with the ability of the user to engage in some or all of their typical activities while the tissue is being treated. Further, many devices, including those that can be donned, do not precisely and effectively direct soothing treatment to the target tissue due to loose and non-specific shape and fit. Further still, many devices are formed with materials that are insulating, causing generalized heating of the entire tissue, when only certain areas are intended for delivery of therapeutic treatment. And some devices, particularly those that are designed to be close fitting, can actually increase discomfort by causing fatigue and possible reduction of blood flow due to poor fit and associated pinching or binding.

These described disadvantages, among others, often discourage use of the assistive devices, resulting in unresolved pain and perhaps slow healing and further deterioration. Thus, the devices in the art may, as a result of their construction or design, be rejected by a user, may fail to provide the desired therapeutic benefit, and also may create the possibility of exacerbating discomfort and pain. Accordingly, there is a need in the art for assistive devices, particularly for extremities such as the hand, which are form fitting to the tissue to ensure targeted delivery of therapeutic treatment to specific tissue, that will remain stably affixed without pinching or binding, and allow for motion and use of the tissue while delivering targeted therapeutic treatment.

### SUMMARY

In a first exemplary embodiment, therapeutic devices include flexible sleeves that include one or more integrated pads, or one or more removable pads, or combinations thereof, where the pads are positioned to direct therapy to isolated portions, such as specific joints of the thumb, hand or wrist, or specific fingers, or specific knuckles, or combinations of these. And in such various embodiments, the pads may be shaped and contoured to cover all or only a portion of the volar, dorsal, medial and lateral surfaces of the anatomy. In various embodiments, each of the one or more pads of a device may deliver the same type of therapy, or different types of therapy, and accordingly, each pad or insert comprising a pad may be formed of the same or different therapeutic materials.

In another exemplary embodiment, the therapeutic pads deliver therapeutic treatment, such as thermal treatment, and more particularly heating, cooling and combinations thereof. Thus, in some embodiments, the therapeutic pads may deliver one or both of heat and cold therapy. In accordance with such embodiments, the pads include one or more materials that can be heated or cooled and retain temperature over a timeframe that allows for delivery of therapeutic benefit either continuously or in cycles that are dependent, in some examples on re-heating or re-cooling the therapeutic material.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description:

FIG. 1 shows a thermal assistive device according to one embodiment;

FIG. 2 shows an alternate view of a thermal assistive device as shown in FIG. 1 having a specific handedness;

FIG. 3 shows an insert for a thermal assistive device as shown in FIG. 1;

FIG. 4 shows a thermal assistive device according to another embodiment;

FIG. 5 shows an alternate perspective view of a thermal assistive device as shown in FIG. 4;

FIG. 6 shows inserts for a thermal assistive device as shown in FIG. 4;

FIG. 7 shows a thermal assistive device according to another embodiment; and

FIG. 8 shows a thermal assistive device according to another embodiment.

Features and advantages of the general inventive concepts will become apparent from the following description made with reference to the accompanying drawings, including drawings represented herein in the attached set of figures, of which the following is a brief description:

### DESCRIPTION

This description provides exemplary embodiments in accordance with the general inventive concepts and is not intended to limit the scope of the invention in any way. Indeed, the invention as described in the specification is broader than and unlimited by the exemplary embodiments set forth herein, and the terms used herein have their full and ordinary meaning.

The general inventive concepts will now be described with occasional reference to the exemplary embodiments of the invention. Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts.

As used herein, the singular forms “a,” “an,” and “the” are intended to include the plural forms as well, unless the context clearly indicates otherwise. Anatomical references as used herein are intended to have the standard meaning for such terms as understood in the medical community. For example, the application may include reference to the following terms: volar (the front, as opposed to the back); dorsal (the back or behind, as opposed to the front); inferior (below, as opposed to superior); superior (above, as opposed to inferior); lateral (toward the left or right side of the body, as opposed to toward the middle); medial (in or toward the middle or inside of the body, as opposed to away from the middle and toward the left or right); proximal (toward the body, as opposed to toward the ends, such as of the fingers and hands); and distal (away from the body, as opposed to towards the body, and towards the ends).

The invention is described herein in relation to amelioration of swelling and pain in joints, including in particular, arthritic conditions of the hand. One skilled in the art will appreciate that the assistive devices and the methods of use thereof, as described herein, can be directed without undue adaptation to applications that include, but are not limited to, remediation of conditions in other tissues and other sites of the body, and amelioration of discomfort associated with a variety of causes, such as fatigue, injury, and post-surgical conditions, and the like.

Generally, in accordance with the embodiments described herein and depicted in the drawings, the invention is directed in various aspects to a contoured assistive device and its components, and methods of use of the assistive device for the relief of joint and soft tissue discomfort.

Assistive Device; Flexible Sleeve and Inserts

As described herein, the assistive device includes an anatomically contoured flexible sleeve that slips over or is fitted and fastened to and closely conforms to the anatomy to be treated, and one or a combination of therapeutic pads that comprise one or more of heating, cooling, and other therapeutic applications. Thus, in such embodiments that include therapeutic pads for delivery of thermal therapeutic applications, the therapeutic pad materials include, but are not limited to, thermal energy storage material, the therapeutic material positioned for targeted treatment of the tissue.

In some embodiments, the assistive device further includes at least one or a plurality of inserts that are positioned or removably positionable in or on the flexible sleeve in targeted areas of the anatomy to be treated, and the inserts including one or more therapeutic pads. In some such embodiments, the inserts can further include one or more retaining elements for retaining therapeutic material. And in some embodiments, the assistive devices can include one or more fasteners to secure the device in place on the anatomy, to retain therapeutic material, to affix an insert to the device, to affix a retaining element in place or in a closed configuration, or combinations of these.

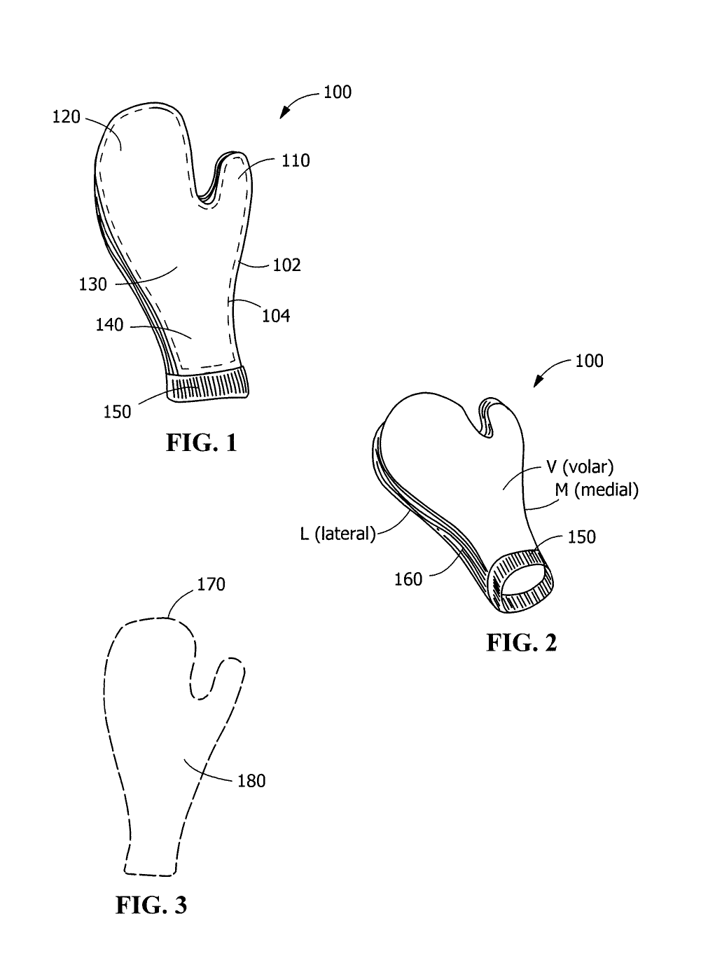

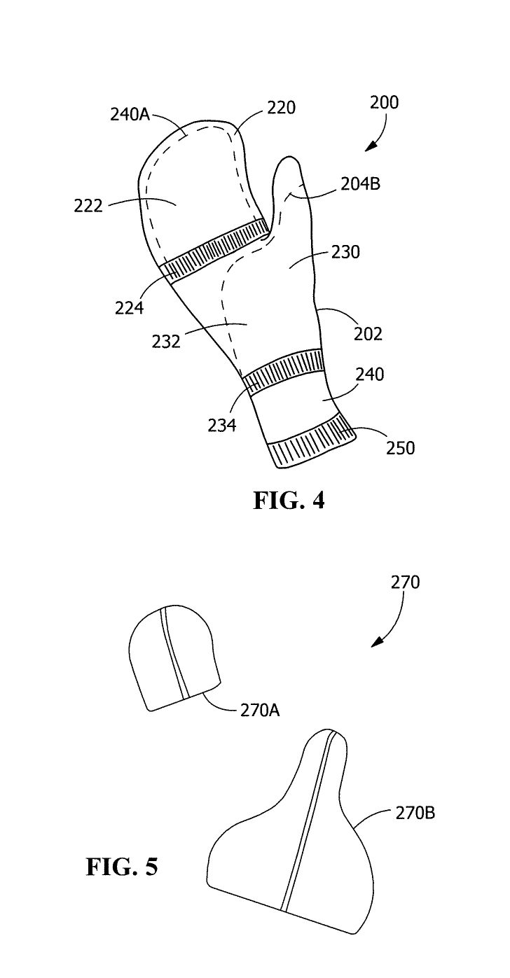

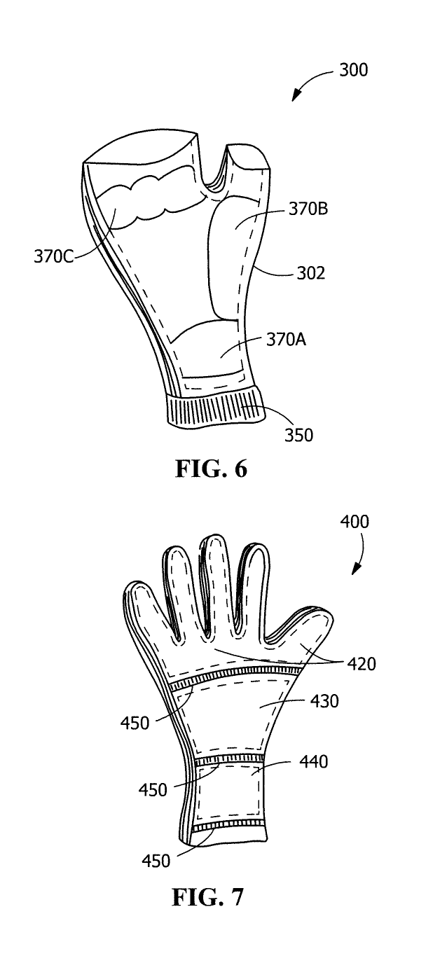

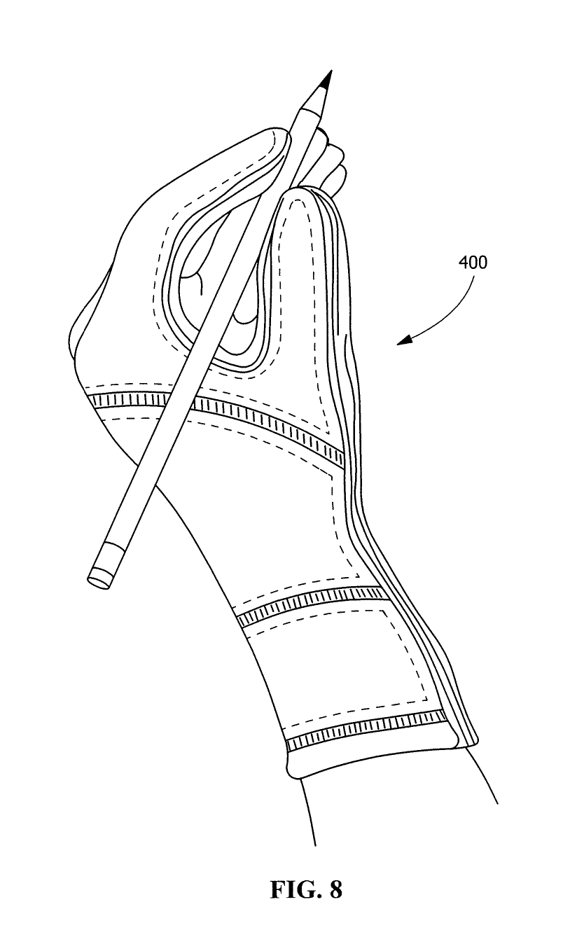

Referring now to the drawings, each of FIG. 1, FIG. 4, FIG. 6 and FIG. 7, shows a different representative embodiment of an assistive device according to the disclosure. Thus, FIG. 1 and FIG. 2 show alternate views of a first embodiment of an assistive device, and FIG. 3 shows an insert suitable for insertion into a sleeve of an assistive device according to FIG. 1 and FIG. 2. Referring again to the drawings, FIG. 4 shows another embodiment of an assistive device, which depicts inserts 270 A and 270 B for insertion into the device of FIG. 4. And FIG. 6 shows yet another embodiment of an assistive device that covers only some portions of the anatomy. In addition, FIG. 7 and FIG. 8 show alternate views of another embodiment of an assistive device.

Referring again to the drawings, as depicted, each of these exemplary embodiments of an assistive device has a profile that is shaped generally like a human hand, with thumb, finger, hand and wrist regions. Referring specifically to FIG. 1, the depicted embodiment of an assistive device 100 includes a flexible sleeve 102 and at least one therapeutic pad 104, and is adapted to fit over the anatomy, in the case of the hand, including a thumb region 110, finger region 120, hand region 130 and wrist region 140.

In accordance with an exemplary embodiment, the assistive device is shaped and configured to fit one or the other of the left or the right hand, for example as shown in FIG. 2, where there are discrete volar V, medial M (the inside edge), lateral L (the outside edge) and dorsal (not shown) sides of the assistive device. In addition, in some embodiments the assistive device is provided in one of a selection of sizes suited to at least small, medium and large hands. Of course, it will be appreciated that in other possible embodiments, any number of sizes and configurations are possible to accommodate a generic range of small, medium and large subjects, or incrementally to accommodate more specific sizes having variable lengths for finger, hand, and wrist sizes.

In accordance with the various embodiments, the assistive device is adapted with features to more precisely accommodate the hand of a user. In some embodiments, referring again to FIG. 1 the assistive device includes a cuff 150 at the wrist for gently securing the assistive device in place. In addition, in some specific embodiments, the cuff 150 may be formed with an elastic material. In other embodiments, the cuff 150 may be non-elastic, elastic, or semi-elastic, and in some embodiments, all or only a portion is elastic or semi-elastic. In some embodiments, the cuff 150 may comprise a belt, band, snap closure, drawstring, or other feature or combinations of these for allowing securement of the assistive device to the user at the wrist, which feature may be in combination with an elastic material.

In accordance with some embodiments, the assistive device is adapted with expansion features that allow expansion to accommodate the size and/or motion of the user's hand. In some examples, these expansion features include one or more of gussets, baffles, crinkled or accordion pleats, and elastic material. Referring now to FIG. 2, the depicted exemplary embodiment includes crinkle pleats 160 around the periphery of the assistive device, where, in the instance of the hand, the assistive device can expand in at least the dimension from volar V to dorsal. In some embodiments, the expansion features are continuous around the entire assistive device, such as in the exemplary embodiments shown in FIG. 1 and FIG. 2, where the pleating is continuous along the edge of the hand. Of course, in other embodiments, the expansion features may be limited to the web between the thumb region 110 and index finger edge of the finger region 120, or to the lateral edge L of the hand, or the medial edge M, or combinations of these.

In the various embodiments, the assistive device is contoured to fit the anatomy to be treated, such as but not limited to the hand. Thus, in some embodiments, the assistive device may be suited for conforming fit to other anatomy, such as, but not limited to, the foot, ankle, wrist, elbow, knee, pelvis, shoulder and neck. And the assistive devices may be adapted for humans or for an animal such as but not limited to a dog. In another exemplary embodiment, not shown, an assistive device fits the foot and/or ankle and is adapted with features as described herein for providing a close fit and custom-targeted therapeutic delivery to anatomical structures.

As shown in the drawings, the assistive devices according to the disclosure are adapted to conform to anatomical areas that include more or fewer specific anatomical structures of the anatomical areas. Thus, as shown in the drawings, for example, FIG. 1, some embodiments of assistive device 100 suited for treatment of the hand is adapted to fit to essentially the full anatomical structure, in this case the hand and wrist, and thus, fit anatomical structures corresponding to the thumb region 110, finger region 120, hand region 130 and wrist region 140. In other embodiments, of course, a device may be adapted to discretely fit each of the individual fingers, in addition to the thumb, or some subsets thereof. Further, in some embodiments, the assistive devices may be adapted to fit or cover only a portion of the anatomy, for example, covering only the portions intended for therapeutic treatment and not covering other structures of the anatomy.

According to some embodiments, the therapeutic pads are supported by the flexible sleeve to direct therapeutic treatment to the target areas and are held in place by portions of the flexible sleeve. Thus, according to such embodiments, in some examples, the therapeutic pads target one or a combination of the distal joints of the fingers, the thumb and the wrist, leaving the distal finger and thumb joints free to allow the user to engage in daily activities. Referring to FIG. 7, in one example, the assistive device 300 does not cover the entire targeted anatomy, as shown in the drawing, wherein the assistive device is adapted to cover only the wrist, hand and proximal portions of the fingers and thumb, leaving exposed the distal portions of the fingers and thumb. In other examples, the devices may include flexible sleeve portions that are limited to coverage of one or a combination of the wrist, the dorsal and/or volar areas of the thumb and hand, and all or a portion of the fingers.

In accordance with an exemplary embodiment according to the invention, the anatomically contoured flexible sleeve is shaped and formed of a flexible mesh or open knit material that moves with the user allowing a close fit without binding. According to one such embodiment, the sleeve is formed at least in part of a flexible and breathable net, weave, or mesh. While in some preferred embodiments, the material includes cotton or one or more additional natural fibers selected from but not limited to linen, hemp, and bamboo, some embodiments may be formed entirely or partially with synthetic and microfiber material. In an exemplary embodiment, the material of the flexible sleeve includes cotton, wicking synthetic fibers, and combinations. In accordance with the various embodiments, the material of the flexible sleeve is non-insulating, ensuring comfort and long wear-ability by the user without developing discomfort due to overheating and sweating.

In some embodiments, the flexible sleeve provides at least localized compression to all or targeted portions of the covered anatomy, which compression may or may not be co-localized with a therapeutic pad. In some embodiments, the compression helps control or decrease swelling, or enhance contact of the therapeutic pad with the targeted tissue, or both.

While the above-described exemplary embodiments as shown in the drawings are adapted for receiving removable inserts that include therapeutic pads, it will be appreciated that in other embodiments, some or all of the pads are integrated with the flexible sleeve and are not removable. According to such embodiments, the pads may be formed partially or entirely of the same material as the flexible sleeve. Thus, in some examples, the flexible sleeve and one or more integrated pads are formed of a material that is flexible and breathable, and can be treated to enable retention and delivery of thermal treatment, such as heating or cooling or both, as further described herein below in relation to the pads.

Pads and Therapeutic Material

In an exemplary embodiment, the therapeutic material is selected from a fluid or particular solid that is conforming and flowable, to allow optimal conforming to the target tissue. In some embodiments, a therapeutic pad is fitted with a flexible sleeve to be in direct contact with the targeted tissue to closely conform to and maximize the direction of thermal benefit to the tissue. In some representative examples, the thermal material may include one or more of gels, ceramic beads, plastic beads, sand and other flowable or formable materials, and heat retaining materials selected from phase change materials (PCMs), for example, sodium polyacrylates, salt hydrates, paraffins, water, vegetable-based PCMs and combinations of these. In some embodiments, the PCMs may be coated onto or formed into flowable fluids or particulates, or combinations of these. PCMs are materials that use phase changes (e.g., solidifying/liquifying) to absorb or release relatively large amounts of latent heat at relatively constant temperature.

In some embodiments, the therapeutic material may be thermally adjusted, whereby the material is either heated or cooled, using any of a variety of methods. In some examples, the material may be cooled using a conventional freezer, or exposure to or immersion in a material such as ice, dry ice, cold water, liquid nitrogen and the like. And in some examples, the material may be cooled using a microwave, a conventional oven, immersion in hot liquid such as hot water and the like, or by application of electrical energy or exposure to another electrified heat generator.

In some embodiments, the pads that are suited for delivery of heat are adapted with features that enable application of electrical energy while donned by the user whereby the therapeutic material may be heated selectively by the user without the need to remove the assistive device.

Thus, in accordance with the foregoing, referring again to FIG. 1, the exemplary assistive device 100 may include two pads 104, one each on the volar and dorsal sides of a sized and handed device, where the pads are either integrated or inserts into the flexible sleeve 102. The assistive device 100 is formed of a cotton mesh material that is open and includes elastomeric threads, wherein the flexible sleeve 102 is close fitting and conforming to the entire hand, and the material is soft, flexible, and breathable and minimizes generalized heating of the hand of the user. The assistive device 100 is adapted with a soft, expandable cuff 150 and with circumferential crinkling pleats 160 that may be elasticized to allow for expansion during movement of the hand, the sleeve's crinkle pleats 160 and cuff 150 both adapted to return to a nominally expanded configuration when the hand is relaxed and a fully non-expanded configuration when the hand is removed. The pads 104 include therapeutic material that is suitable for either or both heating and cooling. According to those embodiments wherein at least one of the pads 104 is provided in an insert 170, the two pads 104 may both be heated or cooled, or one may be heated and the other cooled, as desired by the user. According to those embodiments where both pads 104 are integrated within the flexible sleeve 102 (i.e., they are non-removable) the entire assistive device 100 may be either heated or cooled, including successive rounds of heating, cooling, and combinations of these.

As previously described herein above, in accordance with the various embodiments, the flexible sleeve is adapted with one or more therapeutic pads for targeted anatomical distribution of therapeutic treatment, such as thermal treatment. Referring again to FIG. 3, an exemplary embodiment of an insert 170 comprising a therapeutic pad 180 is shown, wherein the insert 170, now with reference to FIG. 1, is adapted for removable insertion into a receiving pocket (not shown) of a flexible sleeve 102 for positioning of the pad 104 in the sleeve 102. Referring again to FIG. 3, the depicted embodiment of the insert 170 is shaped and sized to cover one of the entire dorsal and volar aspects of the hand and wrist of the user, and in some embodiments, two pads may be used.

Referring again to FIG. 4, yet another exemplary assistive device 200 is shown wherein the flexible sleeve 202, and up to two pads 204 A and one or two of 204 B, are inserts 270 into the flexible sleeve 202. Referring now to FIG. 5, the two inserts 270 A and 270 B, are shown, where the insert 270 A comprises a pad for one or both of the dorsal and volar aspects of the fingers and the insert 270 B comprises a pad for the medial, and partial dorsal and volar aspects of the thumb and wrist. Referring now to FIG. 4, the inserts 270 A and 270 B are adapted for insertion into the correspondingly identified pockets 224 and 234 of the depicted flexible sleeve 202.

The assistive device 200 is formed of a cotton mesh material that is open and includes elastomeric threads, wherein the flexible sleeve is close fitting and conforming to the entire hand, and the material is soft, flexible, and breathable and minimizes generalized heating of the hand of the user. The assistive device 200 is adapted with a soft, expandable cuff 250 that is elasticized to allow for expansion during movement of the hand, cuff 250 is adapted to return to a nominally expanded configuration when the hand is relaxed and a fully non-expanded configuration when the hand is removed. The pads 270 A and 270 B include therapeutic material that is suitable for either or both heating and cooling, wherein the two pads 270 A and 270 B may both be heated or cooled, or one may be heated and the other cooled, as desired by the user.

In yet another exemplary assistive device 300 is shown in FIG. 6, wherein the flexible sleeve 302, and up to three pads 370 A, 370 B, and 370 C, are inserts into the flexible sleeve 302. In some embodiments, assistive device 300 is formed of a cotton mesh material that is open and includes elastomeric threads, wherein the flexible sleeve 302 is close fitting and conforming to the entire hand, leaving the distal portions of the fingers and thumbs free and the material is soft, flexible, and breathable and minimizes generalized heating of the hand of the user. In some embodiments, assistive device 300 is adapted with a soft, expandable cuff 350 that is elasticized to allow for expansion during movement of the hand, cuff 350 is adapted to return to a nominally expanded configuration when the hand is relaxed and a fully non-expanded configuration when the hand is remove. The pad 370 A is adapted as one or two pads that cover at least the volar and dorsal portions of the wrist, and may encircle the wrist and are formed with a therapeutic material that is specifically adapted for one or the other of heating or cooling. Pad 370 B is adapted as one or multiple pad inserts that cover at least the volar, medial and dorsal portions of the thumb, and formed with a therapeutic material that is specifically adapted for one or the other of heating or cooling including therapeutic material that is suitable for either or both heating and cooling. Pad 370 C is adapted as one or multiple pad inserts that cover one or all of the volar, medial, lateral and dorsal portions of the finger joints, and are formed with a therapeutic material that is specifically adapted for one or the other of heating or cooling including therapeutic material that is suitable for either or both heating and cooling. In various uses, as desired by the user, the pads are adapted to provide heating to some locations on the anatomy while others are adapted to provide cooling, and comprise PCM coated ceramic beads, the insert including retaining elements that enclose the therapeutic material to avoid leakage or spillage.

In accordance with the various embodiments, the size and positioning of multiple pad inserts, whether removable or permanent, can continuously cover one or more regions of the hand, wrist and fingers. And in some particular embodiments, as shown herein, the breaks or space between multiple pads can correspond with natural anatomical breaks or joints, thus defining in the assistive device, one or more anatomic or pseudo joints of the device that enable more natural movement of the treated anatomy, particularly the hand.

Referring now to FIG. 7 and FIG. 8, yet another exemplary embodiment is shown. As with the embodiments in FIG. 5 and FIG. 6, the depicted embodiment is adapted for one or more therapeutic inserts to target one or more of the wrist 440, hand 430 and fingers 420. As shown, the depicted embodiment is adapted to retain one or more permanent or removable inserts comprising one or more different therapeutic materials, and also includes breaks or pseudo joints 450 between each of the target areas for therapy.

Referring again to FIG. 7, in some embodiments, the assistive device 400 is adapted with one or more inserts on one or the other of the palmar and dorsal sides, and in some embodiments on both sides, and in yet other embodiments, the assistive device 400 is adapted with different combinations of inserts on both sides. In one example, an embodiment may include or be adapted to insert or have inserted only finger and wrist inserts on the dorsal side, and hand inserts on the palmar side. Yet other combinations are possible. Of course, more generally, in connection with the various embodiments as described herein, the inclusion and placement of therapeutic inserts may vary either in embodiments in which the inserts are fixed, and those in which the user may determine which inserts to include for the targeted tissue.

As described herein with respect to various embodiments, the assistive device 400 is particularly suitable to enable functional use of the treated anatomy, particularly the hand, during treatments. Referring again to the embodiment in FIG. 7 and FIG. 8, the specific contouring and fit to each of the finger 420 digits and the placement of the pseudo joints 450 maximizes the ability to preserve manual dexterity during treatment. In some particular embodiments of the assistive devices, treatment inserts comprising gel or other flowable or formable thermal material enables maximal contoured fit of therapeutic treatment to the desired target zones while allowing nearly full use of the hand.

Use of the Assistive Devices for Therapy

According to the methods hereof, therapy may be delivered by a user without need for medical assistance. In some embodiments, the assistive devices herein may be used to facilitate strategic delivery of one or both cold or heat to all or part of the hand and wrist, for example with a device comprising a flexible sleeve 102 according to FIG. 1 that includes two built-in thermal pads. The thermal pads can be cooled in any freezer for cold application or placed in a microwave to provide heat. In use, typical application cycles of about 20 minutes are suitable for cold application, and can be repeated as often as needed, with 20 minute rest intervals. In some embodiments, heat may be applied continually. The assistive devices may be indicated for use as an adjunctive therapy for the hand and wrist after injury or surgery, and for management of arthritic symptoms. In exemplary embodiments, the assistive devices include flexible sleeves that are made with soft breathable cotton to keep skin dry, and provide mild compression to help control or decrease swelling.

Significant benefits can be realized in connection with therapeutic use of the assistive devices hereof, including, but not limited to: optimized patient experience and outcome as a result of controlled and precise application of therapeutic treatment to the anatomy to reduce swelling and provide pain relief and possible reduced reliance on drug therapeutics. Unless otherwise indicated, all numbers expressing quantities, properties, and so forth as used in the specification and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that any numerical ranges and parameters setting forth the broad scope of the general inventive concepts are approximations, numerical values set forth in the specific examples are reported as precisely as possible. Any numerical values, however, inherently contain certain errors necessarily resulting from error found in their respective measurements.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts.

Those skilled in the art may readily adopt one or more of the inventive aspects, concepts or features into additional embodiments and uses within the scope of the general inventive concepts even if such embodiments are not expressly disclosed herein. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated. Further, while disclosed benefits, advantages, and solutions to problems have been described with reference to specific embodiments, these are not intended to be construed as essential or necessary to the invention.

1. An anatomically contoured assistive device comprising
a flexible sleeve, and
at least one therapeutic pad
the flexible sleeve comprising at least one segment that is contoured to fit at least a portion of an anatomical structure, and formed of a flexible and breathable material, and adapted to retain the at least one therapeutic pad
the therapeutic pad comprising one or a combination of therapeutic treatment materials, the pad being conformable to all or a part of the anatomical structure.

2. An anatomically contoured assistive device according to claim 1, wherein the sleeve slips over or is fitted and fastened to and closely conforms to the anatomy to be treated.

3. An anatomically contoured assistive device according to claim 1, wherein each of the at least one therapeutic pads comprises one or a combination of therapeutic materials selected from thermal energy storage phase change materials.

4. An anatomically contoured assistive device according to claim 1, the therapeutic material positioned adjacent to the flexible sleeve for targeted treatment of at least one select anatomical structure.

5. An anatomically contoured assistive device according to claim 1, the assistive device further comprising at least one or a plurality of inserts comprising one or more pads, the inserts removably positionable in or on the flexible sleeve.

6. An anatomically contoured assistive device according to claim 1, at least one pad comprising one or more retaining elements for retaining therapeutic material.

7. An anatomically contoured assistive device according to claim 1, further comprising one or more fasteners that secure the device in place when donned, retain therapeutic material within an insert or retaining element, affix an insert to the device, to affix a retaining element in place or in a closed configuration, or combinations of these.
